(19) United States
(12) Patent Application Publication
(10) Pub. No.: US 2015/0272509 A1
(43) Pub. Date: Oct. 1, 2015

(54) DIAGNOSTIC APPARATUS AND METHOD

(71) Applicant: Samsung Electronics Co., Ltd., Suwon-si (KR)

(72) Inventor: Keun Joo KWON, Seoul (KR)

(73) Assignee: SAMSUNG ELECTRONICS CO., LTD., Suwon-si (KR)

(21) Appl. No.: 14/524,741

(22) Filed: Oct. 27, 2014

(30) Foreign Application Priority Data
Mar. 31, 2014 (KR) 10-2014-0038074

(51) Int. Cl. A61B 5/00; A61B 5/0464; G06F 19/00; G06N 99/00; G06N 7/00; A61B 5/0452; A61B 5/046

(52) U.S. Cl. CPC A61B 5/7267; A61B 5/0452; A61B 5/0464; A61B 5/046; A61B 5/7278; G06N 99/005; G06N 7/005; G06F 19/345

(57) ABSTRACT

A diagnostic apparatus and method are described. The diagnostic apparatus includes a diagnostic model unit configured to diagnose time-series data based on a model structure and parameters of a diagnostic model performing probability model-based analysis. The diagnostic apparatus also includes a learner configured to change the parameters using the time-series data as training data, and a change detector configured to detect a parameter change and output an alarm signal based on the detected parameter change.

### CROSS-REFERENCE TO RELATED APPLICATION(S)

This application claims the benefit under 35 U.S.C. §119(a) of Korean Patent Application No. 10-2014-0038074, filed on Mar. 31, 2014, in the Korean Intellectual Property Office, the entire disclosure of which is incorporated herein by reference for all purposes.

### BACKGROUND

1. Field

The following description relates to a diagnostic technique, and more particularly, to a diagnostic technique using a diagnostic model that updates parameters by online learning.

2. Description of the Related Art

In general, an online learning technique to enhance performance of a diagnostic model receives time-series data and statistically analyzes the time-series data based on a diagnostic model to produce a diagnostic result.

For example, a diagnostic system to diagnose heart failure receives data, such as electrocardiography (ECG) data, which is obtained from a patient, and diagnoses a state of heart failure of the patient using a diagnostic model, such as Hidden Markov Model (HMM). In principle, the diagnostic models are based on stationary distributions described by predefined parameters. For example, based on a predefined diagnostic model, a diagnostic system generates a normal distribution from ECG data input during a predetermined period of time, such as during the last one minute of running the predefined diagnostic model, and extracts variables, such as an average or a variance of the normal distribution. Then, the extracted variables are analyzed based on the predefined parameters, so that a diagnostic result indicating a state of the heart failure of the patient may be inferred.

Performance of a diagnostic system adapting a specific diagnostic model may be determined based on whether a parameter of the specific diagnostic model is defined well to describe input data. In general, the parameter of the diagnostic model may be defined through a learning process performed using a pre-stored training data. As the learning process requires repetitive computations that require a relatively enormous amount of training data, the learning process is commonly a pre-learning type which is done before an actual diagnostic procedure begins.

Unlike the pre-learning type, an online learning technique is a technique of adjusting a parameter of a diagnostic model, in real time, based on input data. In the above example, a parameter of the current diagnostic model is adjusted, in real time, using a patient's ECG data being detected as a training data. The online learning technique continuously changes a parameter or parameters of a diagnostic model during the entire diagnostic procedure. A diagnostic model includes at least one parameter. The online learning technique makes it possible to personalize a diagnostic model. However, while the online learning technique is used, previously online learned results on previous input data disappears, because the online learning technique changes the diagnostic parameters based on the currently input data. For this reason, a diagnostic device with parameters changed using the online learning technique may output an unwanted result.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In accordance with an illustrative example, there is provided a diagnostic apparatus, including a diagnostic model unit configured to diagnose time-series data based on a model structure and parameters of a diagnostic model performing probability model-based analysis; a learner configured to change the parameters using the time-series data as training data; and a change detector configured to detect a parameter change and output an alarm signal based on the detected parameter change.

The change detector may also include a receiver configured to receive a parameter value of a parameter, a change determiner configured to compare the parameter value with a pre-stored reference parameter value and determine whether the parameter has changed based on a difference between the parameter value and the pre-stored reference parameter value, and an output configured to output the alarm signal in response to a determination that the parameter has changed.

The pre-stored reference parameter value may be identical to a default parameter value, which is pre-set prior to the learner performing online learning on the parameter.

In response to the parameter value changing from a default parameter set before the learner performs online learning, the change determiner may be further configured to determine that the parameter changes to be greater than a predetermined value, in a constant direction for a predetermined period of time, or at a speed or acceleration greater than a predetermined level.

The parameter value may gradually or suddenly change from a default value or an initial value through online learning.

The change detector may also include a receiver configured to receive the parameter value, a distribution generator configured to generate a probability distribution of the parameter value, a change determiner configured to determine whether a probability distribution of the parameter value has changed based on a difference between a distribution value indicative of properties of the probability distribution of the parameter value and a distribution value indicative of properties of a probability distribution of the reference parameter value, and an output configured to output the alarm signal in response to a determination that the probability distribution of the parameter value has changed.

The probability distribution of the reference parameter value may be identical to a probability distribution of a default parameter value pre-set before the learner performs online learning.

In response to a mean or a variance of the probability distribution of the parameter value changing from a mean or a variance of the probability distribution of the default parameter value more than a predetermined value, the change determiner may be further configured to determine that the probability distribution of the parameter value changes in a constant direction for a predetermined period of time, or at a speed greater than a predetermined level.

The diagnostic model unit may be further configured to perform diagnosis based on a model structure configured to include hidden nodes and observable nodes, and parameters, each including a conditional transition probability for a default distribution of the hidden nodes relative to time and a conditional output probability for relations between the hidden nodes and the observable nodes.

The diagnostic model unit may be configured to receive electrocardiography (ECG) signals detected from a subject of observation as time-series data and output a diagnostic result estimating or predicting a heart disease of the subject based on the received ECG signals, a value of the observable nodes includes a raw ECG signal, a value converted from the raw ECG signal, and a value extracted from the ECG raw signal, and values of the hidden nodes includes values indicative of a heart condition, a value indicative of medical significance presented on ECG, and a state of a body condition, wherein the value indicative of the heart condition includes atrial systole, complete atrial systole, ventricular systole, complete ventricular systole, ventricular diastolic relaxation, and diastasis, and the body condition includes an increasing heart rate, a decreasing heart rate, a high heart rate, a low heart rate, and a stable heart rate.

The time-series data may be transmitted from a remote device to the diagnostic model unit over a communication network, or a diagnostic result output from the diagnostic model unit is transmitted to the remote device over the communication network.

Other features and aspects may be apparent from the following detailed description, the drawings, and the claims.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram illustrating an embodiment of a diagnostic apparatus.

FIG. 2 is a block diagram illustrating an embodiment of a change detector shown in FIG. 1.

FIG. 3 is a block diagram illustrating another embodiment of a change detector shown in FIG. 1.

FIG. 4 is a block diagram illustrating another embodiment of a change detector shown in FIG. 1.

FIG. 5 is a block diagram illustrating another embodiment of a diagnostic apparatus.

FIG. 6 is a block diagram illustrating another embodiment of a diagnostic apparatus.

FIG. 7 is a block diagram illustrating another embodiment of a diagnostic apparatus.

FIG. 8 is a flow chart illustrating an embodiment of a diagnostic method.

FIG. 9 is a flow chart illustrating an embodiment of a method, shown in FIG. 8, in which a parameter change is detected.

FIG. 10 is a flow chart illustrating another embodiment of a method, shown in FIG. 8, in which a parameter change is detected.

FIG. 11 is a flow chart illustrating another embodiment of a method, shown in FIG. 8, in which a parameter change is detected.

FIG. 12 is a flow chart illustrating another embodiment of a diagnostic method.

FIG. 13 is a flow chart illustrating another embodiment of a diagnostic method.

Throughout the drawings and the detailed description, unless otherwise described, the same reference numbers will be understood to refer to the same elements, features, and structures. In the figures, the left-most digit(s) of a reference number identifies the figure in which the reference number first appears. The relative size and depiction of these elements may be exaggerated for clarity, illustration, and convenience.

### DETAILED DESCRIPTION

The following description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

Time-series data is data that is detected in a successive manner during a period of time. The time-series data may include data from various applications, such as biological data detected or captured from a human body for a purpose of diagnosis of a disease, monitoring data to detect faulty components in a plant's machine or in an automobile, and environmental data necessary for weather forecasts or seismological observation relating to humidity, temperature, vibration, and the like.

The time-series data, for example, in the case of biological signals, may be obtained by automatically measuring data by applying a measuring sensor to a subject of observation, such as a human patient, or by periodically measuring manually the data. Such biological signals may include electrocardiography (ECG) data, body temperature measurement data, blood analytic data, and oxygen saturation measurement data, and the like.

Herein, embodiments are described using a diagnostic model for analyzing ECG signals, that is, ECG data, in order to diagnose a patient with heart failure. However, a person of ordinary skill in the art will appreciate that other types of data, such as the data previously discussed, and other applications, such as automotive diagnostics may be used to analyze a variety of time-series data.

For a technique that obtains an estimated or expected diagnostic result from time-series data using a diagnostic model for statistical analysis, an object of interest that can be modeled may be included as an analysis-subjected object, among objects of interest. In one example, an object of interest may be diagnosed using a diagnostic model only when the object of interest may be modeled by the diagnostic model. Practically, it is impossible to model all objects of interest in the real world. An object may be more meaningful and more important when it is impossible to determine the object properly using a diagnostic model than when the object is determined using a diagnostic model. In order to overcome the limitations of diagnosis using a diagnostic model, a technique, such as an error detection technique, has been developed.

An error detection technique is a technique of detecting whether an error of a diagnostic result output from a diagnostic model is within a predetermined range. The error detection technique may use, for example, a cumulative sum (CUSUM) algorithm, Kalman filter or the like. The error detection technique enables detecting whether an error in a diagnostic model strays away from a predetermined range, and, if so, recognizing an error state, rather than a normal state. If the error state is recognized, a corresponding diagnostic result may be neglected because a meaning of the error state may not be possible to recognize. Alternatively, a measurement, for example, adjusting a parameter of a diagnostic model to include the error state in diagnostic results, may be taken, but it is possible only when numerous identical or similar error states occurs and when the meaning of the error state is able to be interpreted.

Meanwhile, an online learning technique is a technique for minimizing the magnitude of an error in a diagnostic model. With respect to an arbitrary model, it is desirable for parameters to be set as values that describe data to be input to the model, and for this purpose a parameter setting process is performed through learning. In general, learning is performed through pre-learning procedure in which parameters of a model are adjusted using prepared or predefined training data. On the other hand, online learning is a procedure of adjusting parameters of a model online using data that continue to be input to the model during operation thereof.

As such, online learning continues to update parameters of a model online using input data, so the parameters may be adapted to describe the input data the most precisely. It helps to personalize a diagnostic model for each patient. Online learning is advantageous in a medical diagnosis field in which a disease is diagnosed based on data that vary from person to person.

However, in an online learning diagnostic model, parameters continue to be learned using a most current data, so that online learning makes the information previously generated disappear. In this procedure, a diagnostic model is transformed by the online learning into a model different from a previous diagnostic model, possibly leading to knowledge corruption in which a result may be output that is different from what was initially intended.

In other words, in the online learned diagnostic model, when a previous parameter value is changed to be a current parameter value, the previous parameter value disappears or is deleted. For this reason, when an object of interest is a change between diagnostic results collected during a long period of time or during a long term, it is hard to apply an online diagnostic model.

For example, a diagnostic model generates a diagnostic result from a patient with heart failure based on change of ECG data collected for a relatively short term. However, based on a short term change, it is difficult for the diagnostic model to identify a threshold, that is, a point in time indicating the transition from a relatively healthy state to a relatively un-healthy state at which symptoms of heart failure the patient requires aggressive medical treatment. Identifying the threshold is more difficult when the patient is at an early stage of a disease because the symptomatic difference between a health state and a un-health state may not be well recognized or identified by the diagnostic model. Treatment is most effective when it is done at an early stage of a disease. Thus, for medical diagnosis, it is critical to detect a threshold to transition from a healthy condition to an early-stage disease.

In a diagnostic technique using an online learning diagnostic model, parameters of a diagnostic model may be changed from initial values through online learning. An initial value of a parameter value may be a learned value using average data obtained from healthy people within an age range, gender, race, and other physical or mental conditions as the patient. A changed value of a parameter may be a learned value of the initial value using data obtained from a specific individual, who is an object of interest, during a specific period of time. Thus, each of all the parameter values between a default parameter value and a current parameter value may be considered to be an indicator that describes a state of a specific individual during a specific period of time. Further each difference between all the parameter values and a parameter default value may be considered as an indicator, which describes a difference between states of an average healthy person and a specific individual being diagnosed at a specific period of time.

Thus, in an online learning diagnostic model, it is possible to detect, for each parameter, how much a parameter value has changed from a previous parameter value to the current parameter value, to obtain a diagnostic result that is based on a relative long-term change between a previous state and the current state. The diagnostic result based on the long term change may provide important resources that could determine a critical point of the parameter indicative of a transition of the patient from a healthy state to a un-healthy state, that is, a point in time where treatment should be started.

According to embodiments, a diagnostic apparatus and method change parameters of a diagnostic model through online learning and monitor a change over time of parameters. As a result, the diagnostic apparatus and method provides a personalized diagnostic device that obtains a diagnostic result optimized for an individual through online learning. The diagnostic apparatus and method also provide a diagnostic technique of obtaining a diagnostic result based on a relatively long-term change using a diagnostic model that obtains a diagnostic result based on a relatively short-term change.

In addition, according to embodiments, the diagnostic apparatus and method change a parameter value of a diagnostic model through online learning, and detect various states of changes, such as change of a parameter value or a change of a value indicative of properties of a distribution of the parameter value. The states of changes detected include, but are not limited to, amounts of the changes, directions of the changes, and speeds of the changes and accelerations of the changes. By detecting amounts of the changes, the diagnostic apparatus and method detect whether a parameter value or a value indicative of properties of a corresponding distribution has increased or decreased from an initial value. By detecting directions of the changes, the diagnostic apparatus and method detect whether the direction of the change of a parameter value or the change a value indicative of properties of a corresponding distribution has occurred to an increased or decreased direction at a corresponding point. In addition, by detecting speeds or accelerations in the changes, the diagnostic apparatus and method detect whether the change of a parameter value or the change of a value indicative of properties of the parameter distribution has been occurred slowly or rapidly. Accordingly, the diagnostic apparatus and method are configured to provide a personalized diagnostic device that produces a diagnostic result optimized for an individual through online learning, but also to provide a diagnostic technique that produces a diagnostic result based on various aspects of the parameter change made by online learning.

According to embodiments, the diagnostic apparatus and method change a parameter value of a diagnostic model through online learning and detect a parameter change to determine that the data distribution assumed in the diagnostic model is a non-stationary distribution, rather than a stationary distribution, based on the detected parameter change.

In general, one of the assumptions in model-based data analysis is that data distributions are stationary distributions. In other words, a diagnostic device performing data analysis based on a diagnostic model performs the diagnosis assuming that an input data distribution is consistent with a predefined stationary distribution. When the distribution of input data is not a predetermined stationary distribution but a non-stationary distribution, the diagnostic device may not be able to perform a diagnostic determination and an error state may occur. However, in a case of a diagnostic device in which a parameter is changed through online learning, input data is used as training data to adjust a stationary distribution that is predefined by a diagnostic model using the parameter. Because it is hard to determine whether a diagnostic model including a parameter learned online has a non-stationary input data distribution, knowledge corruption could occur. Accordingly, in order to avoid knowledge corruption and detect an error state, it is necessary to detect a change in a diagnostic model.

In embodiments, detection of a parameter change enables detection of a change in a diagnostic model. A change of the diagnostic model's parameter indicates that a data distribution currently defined by the diagnostic model is non-stationary distribution, deviated from the stationary data distribution previously defined by the diagnostic model. As such, according to embodiments, a diagnostic technology generates a personalized diagnostic model to obtain a diagnostic result optimized for an individual, and detects a change of the diagnostic model to discover an error state in which the diagnostic model is unable to determine the situation.

The diagnostic apparatus and method, in accord with embodiments, are configured to detect a parameter change in a diagnostic model; thus, providing multiple advantages and benefits. For example, the diagnostic apparatus and method, according to embodiments, are not required to assume that the diagnostic model has changed. In addition, the diagnostic apparatus and method model does not require an error state for detection and secure training data indicative of the error state for learning a parameter. The diagnostic apparatus and method, according to embodiments, enable detecting an error state using a diagnostic model with a parameter learned using training data indicative of a normal state.

In a case of diagnosis of, for example, a heart disease, the diagnostic apparatus and method, according to an embodiment, detects a parameter change in a diagnostic model that is a probability model required for diagnosis of the disease, thereby enabling a medically significant determination, such as prognosis of a heart failure and prediction of a heart attack, which is hard to obtain based on measurable data, for example, a heart rate.

Hereinafter, diagnostic systems and methods according to embodiments are illustratively described with reference to drawings.

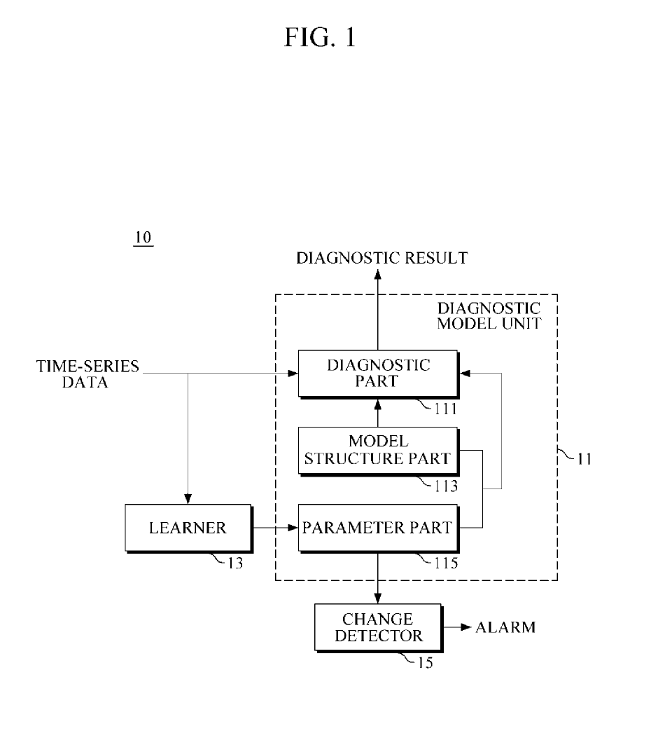

With reference to FIGS. 1 to 7, embodiments of diagnostic apparatus are described. However, the diagnostic apparatus described with reference to FIGS. 1 to 7 are merely descriptive and exemplary. It is apparent for those skilled in the art that different diagnostic apparatuses with various combinations are possible within the scope of the following embodiments. Components of a diagnostic apparatus are implemented by hardware that includes structural devices, elements, and circuits enabling functions for the respective components.

FIG. 1 is a block diagram illustrating an embodiment of a diagnostic apparatus.

Referring to FIG. 1, in a diagnostic model with parameters that may be changed by online learning, there is provided an example of a diagnostic apparatus 10 in which parameter changes are detected.

The diagnostic apparatus 10 includes components such as a diagnostic model unit 11, a learner 13, and a change detector 15. In one configuration, the learner 13 and/or the change detector 15 may be external to the diagnostic apparatus 10, which would include the diagnostic model unit 11. In an alternative configuration, the diagnostic model unit 11, the learner 13, and the change detector 15 may be integral to the diagnostic apparatus 10.

The diagnostic model unit 11 is a structural component that performs analysis on input data based on a model to generate a diagnostic result, and operates based on a probability model. The diagnostic model unit 11 receives time-series data, performs diagnosis on the time-series data based on pre-stored model structures and parameters, and outputs a diagnostic result.

The diagnostic model unit 11 includes a diagnostic part 111, a model structure part 113, and a parameter part 115. The parts described herein are implemented using hardware components. The hardware components may include, for example, controllers, sensors, processors, generators, drivers, and other equivalent electronic components.

The diagnostic part 111 performs diagnosis based on a model structure pre-stored in the model structure part 113 and a parameter pre-stored in the parameter part 115. The parameter of the parameter part 115 is set based on the model structure of the model structure part 113. The model structure in the model structure part 113 is a structure of any one of various probability models, and the parameter in the parameter part 115 is a criterion or a condition used to obtain a diagnostic result in accordance with the model structure in the model structure part 113.

Take an example in which the diagnostic model unit 11 employs a probability model, such as Hidden Markov Model (HMM). HMM is a time-series model with a model structure including observable nodes and hidden nodes. In this case, the model structure 113 includes a structure indicative of a correlation between the observable nodes and the hidden nodes. The hidden nodes have the Markov property in which the hidden nodes depend upon a state of the hidden nodes of the previous time unit, but have nothing to do with other time units. The observable nodes depend upon hidden nodes of an identical time unit. And, in this example, the parameter part 115 includes as parameters “conditional transition probabilities” between initial distributions and initial time of hidden nodes and “conditional output probabilities” between hidden nodes and observational nodes.

The initial values of the parameter part 115 are values learned using a predefined training data. For example, in a case of diagnosis of a heart disease based on time-series data, such as ECG data, initial values of the parameter part 115 of the diagnostic model unit 11 are set using a training data, which is a collection of ECG data measured from average healthy people.

The learner 13 learns the initial values of the parameter part 115 through online learning and while the diagnostic model unit 11 is performing diagnosis. The initial values of the parameter part 115 are adjusted to values personalized for a specific individual. For example, when a health condition of a person with a heart disease is gradually changed, ECG data of the person is also changed. As a result, parameters learned from the ECG data, through online learning, gradually changed over time.

The diagnostic part 111 is a processing structural component that performs analysis, prediction or estimation on time-series input data based on the model structure and the parameters. A value predicted or estimated by the diagnostic part 111 is output from the diagnostic model unit 11 as a diagnostic result.

The learner 13 is an online learning structural component that changes parameters stored in the diagnostic model unit 11 by performing real-time learning processes using the time-series data input to the diagnostic model unit 11. Due to the online learning, parameters of the parameter part 115 of the diagnostic model unit 11 are changed to the current values from initial values.

If, for example, the diagnostic model unit 11 operates based on a diagnostic model designed for diagnosis of a heart disease, initial values of parameters are determined by learning in advance using a training data that is a collection of data obtained from healthy people. Then, the diagnostic model unit 11 receives ECG data detected from a specific individual, who is a subject of observation, as input data, and performs diagnosis to identify, for example, a heart disease. In turn, the learner 13 uses the currently received ECG data as training data to change the initial values of the parameters of the diagnostic model unit 11 into the current values thereof. The diagnostic model unit 11 performs diagnosis on data received at a present time or a next time using a parameter changed at the present time through online learning. In this manner, a diagnostic model employed by the diagnostic model unit 11 is adjusted to become a personalized diagnostic model optimized for a specific individual.

A well-known online learning technique for online learning of parameters of a diagnostic model may be used, for example, numerical analysis, recursive estimation, or the like.

The change detector 15 is a structural component that detects changes of the parameters in the diagnostic model unit 11 and outputs a change detection signal in response to the detected changes of the parameters exceeding predetermined thresholds. The diagnostic model unit 11 detects a change in time-series data observed from a specific object or patient, and outputs a predetermined diagnostic result in accordance with a degree of the detected change. On the other hand, the change detector 115 detect a change of a parameter from the diagnostic model unit 11 and outputs an indirect diagnostic result which carries significance but is unable to be diagnosed by the diagnostic model unit 11 in accordance with a degree of the detected parameter change.

A parameter value may be gradually or suddenly changed from a default value or an initial value by online learning. The change detector 15 detects a change over time or a sudden change of the parameter value. Alternatively, the change detector 15 calculates a probability distribution of the parameter value and a value, such as a mean and a variance, which is indicative of properties of the probability distribution, and detects a change of the calculated value over time. Hereinafter, a “distribution value” refers to a mean or a variance which is indicative of properties of a distribution.

In addition, the change detector 15 detects whether the amount of change in a parameter value or a parameter distribution value has been reached. That is, by determining whether the amount of change in a parameter value or a distribution value of a parameter is greater than a predetermined threshold, the change detector 15 detects a change in the parameter value or the parameter distribution value. For example, in a case where a parameter value or a parameter distribution value increases or decreases by more than 5% or 10%, the change detector 15 determines that a corresponding parameter has changed. Further, the change detector 15 may detect states of change of a parameter value or of a parameter distribution value, for example, such as direction, speed or acceleration in the change.

In other words, the change detector 15 detects an event where a default or initial value of a parameter has been changed to an extent greater than a predetermined value, or where the values of parameter are gradually increased, gradually decreased, or rapidly changed. In addition, the change detector 15 detects an event where an initial mean value or an initial variance value of a parameter distribution has been changed to an extent greater than a predetermined value, or where the mean values or the variance values are gradually increased, gradually decreased, or rapidly changed. The change detector 15 may employ any one of various change detection algorithms. For example, the change detector 15 may employ any one of a cumulative sum (CUSUM) algorithm, a generalized CUSUM algorithm or the like.

An alarm signal output from the change detector 15 is a signal notifying detection of a change of a parameter, and may further include additional data. For example, the alarm signal may further include additional data, such as data to identify a parameter, data to identify a changed parameter from among a plurality of parameters, and/or data indicative of time length between a detection point in time of a changed parameter and a beginning point in time of diagnosis.

The diagnostic model unit 11 and the change detector 15 may operate independently. For example, in a case where a diagnostic result output from the diagnostic model unit 11 shows a normal state, the change detector 15 outputs an alarm signal in response to the detection of parameter change. The alarm signal output from the change detector 15 may indicate that a parameter of the diagnostic model unit 11 has been changed from a default value to a significant extent. An operator of the diagnostic apparatus 10 may not be able to identify the meaning of the received alarm signal, but at least recognize the fact that the alarm signal indicates a change whereby a subject of observation, such as a person or an animal, is in a condition requiring a certain treatment.

In addition, an alarm signal output from the change detector 15 may indicate that a personalized diagnostic model of the diagnostic model unit 11 has changed too much through online learning, compared to an originally-intended diagnostic model. That is, an alarm signal may indicate that the data distribution assumed by the current diagnostic mode changed by online learning is non-stationary, rather than a stationary data distribution, which is assumed by a previous diagnostic model that has yet to be changed through online learning. Therefore, even in a case where a diagnostic result output from the diagnostic model unit 11 does not indicate an abnormal state, and the change detector 15 outputs an alarm signal, a user who receives the alarm signal may recognize that a subject of observation is in an abnormal condition requiring a certain treatment.

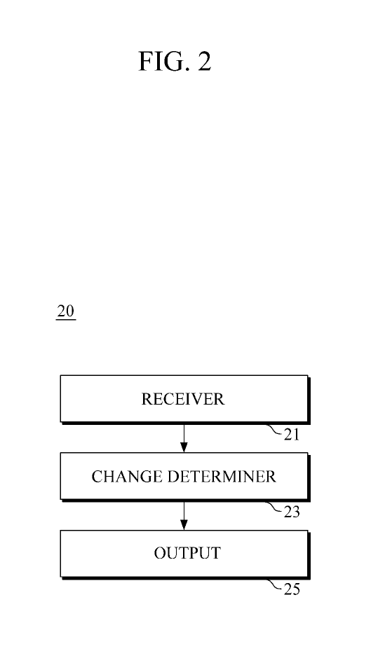

Hereinafter, the change detector 15 of the diagnostic system 10 shown in FIG. 1 is described in greater detail with reference to FIGS. 2 to 4.

FIG. 2 is a block diagram illustrating an embodiment of a change detector 15 illustrated in FIG. 1 and previously described.

Referring to FIG. 2, a change detector 20 is a structural component that detects changes of parameter values, and includes structural components such as a receiver 21, a change determiner 23, and an output 25 or the like. A person of ordinary skill in the art will appreciate that additional structural components may be included in the change detector 20.

The receiver 21 is a structural component that receives values of a parameter from the parameter part 115 of the diagnostic model unit 11 shown in FIG. 1.

The change determiner 23 compares a current parameter value received at the receiver 21 to a reference parameter value pre-stored in the change detector 20. After comparison, the change determiner 23 determines whether the current parameter has changed based on a difference between the current parameter value and the reference parameter value.

In one illustrative example, the reference parameter value may be identical to a default parameter value that was set before a corresponding parameter changed through online learning.

The change determiner 23 determines change of a parameter in various ways. For example, in a case where the current parameter value has changed from a reference parameter value, such as a default parameter value, by a predetermined amount, the change determiner 23 determines that the parameter has changed. Further, in a case where the current parameter value has changed in a constant direction for a predetermined period of time, the change determiner 23 determines that the parameter has changed. For example, in a case where values of a parameter of a diagnostic model has increased or decreased for three consecutive months in a diagnostic system for diagnosing a heart disease based on received ECG signals, the change determiner 23 determines that the parameter has changed. Further, in a case where values of a parameter change at a rate greater than a predetermined value, the change determiner 23 determines that the parameter has changed. The rate of change of the values of a parameter may enable determining whether the parameter has been gradually increased, gradually decreased, rapidly increased or rapidly decreased. In addition, the change determiner 23 determines a parameter change by observing acceleration in change of the values of the parameter, rather than speed in the change.

The output 25 is a component that outputs an alarm signal in response to the change determiner 23 determining that a parameter has changed. The alarm signal output from the output 25 may be a sound signal or a visual signal notifying that a change of a specific parameter is detected. In such a case, a user who has received the alarm signal may not be able to identify the exact meaning of the alarm signal, but able to recognize at least the fact that a subject, such as a person or an animal, of observation is in a condition requiring a certain treatment.

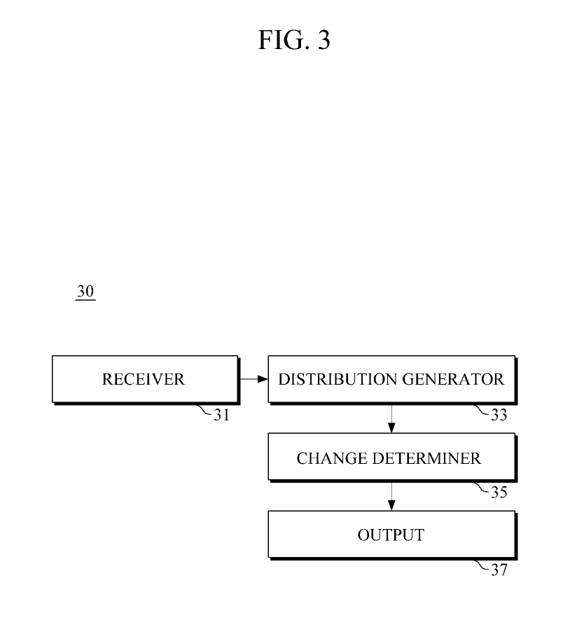

FIG. 3 is a block diagram illustrating another embodiment of a change detector 15 shown in FIG. 1 and described above.

Referring to FIG. 3, a change detector 30 is a structural component that detects a change in probability distributions of a parameter, and includes structural components such as a receiver 31, a distribution generator 33, a change determiner 35, and an output 37. In one alternative configuration, the receiver 31 may be external to the change detector 30. Furthermore, in another alternative configuration, additional structural components may be included in the change detector 30 in addition to those illustrated in FIG. 3.

The receiver 31 is a structural component that receives values of a parameter from the parameter part 115 of the diagnostic model unit 11 shown in FIG. 1.

The distribution generator 33 generates a probability distribution of the current parameter value.

The change determiner 35 compares a distribution value indicative of properties of the probability distribution, generated at the distribution generator 33, of the current parameter value to a distribution value indicative of properties of a probability distribution of a reference parameter value pre-stored in the change detector 30. Through the comparison, the change determiner 35 determines whether a probability distribution of the current parameter value has been changed based on a differential between a distribution value of the current parameter and a distribution value of the reference parameter.

In one illustrative example, the probability distribution of the reference parameter value is identical to the probability distribution of the initial or parameter value set before a change in a corresponding parameter through online learning.

The change determiner 35 determines a change of a probability distribution of a parameter in various ways. In one illustrative example, in a case where the current distribution value of the parameter has been changed more than a predetermined amount from a reference distribution value of the parameter, that is, a default or initial distribution value, the change determiner 35 determines that the probability distribution of the parameter has changed. In another illustrative example, in a case where the distribution value of the parameter has changed in a constant direction for a predetermined period of time, the change determiner 35 determines that the probability distribution of the parameter has changed. For example, in a diagnostic system receiving ECG signals and diagnosing heart diseases, if the distribution value of a parameter of a diagnostic model in the diagnostic system has increased or decreased for the three-consecutive months, the change determiner 35 determines that the probability distribution of the parameter has changed. In a further illustrative example, in a case where the rate of change of the distribution value of the parameter is greater than a predetermined value, the change determiner 35 determines that the probability distribution of the parameter has changed. The speed in a change of a distribution value of a parameter may enable determining that a probability distribution of the parameter has slowly increased/decreased, or rapidly increased/decreased. Moreover, the change determiner 23 determines whether a probability distribution of a parameter has changed, by observing acceleration in change of the distribution value of the parameter, rather than speed in the change.

The output 37 is a structural component that outputs an alarm signal when the change determiner 35 determines that a probability distribution of a parameter has changed.

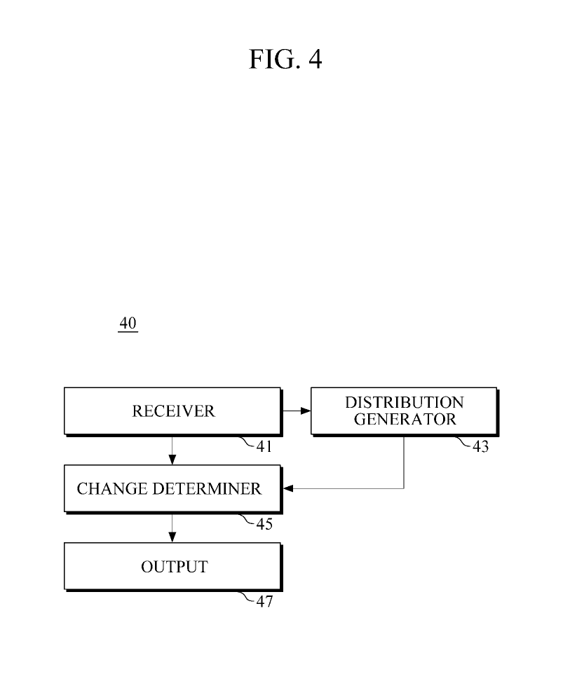

FIG. 4 is a block diagram illustrating another embodiment of a change detector 15 shown in FIG. 1.

Referring to FIG. 4, a change detector 40 is an example of a combination between the change detector 20 shown in FIG. 2 and the change detector 30 shown in FIG. 3. The change detector 40 is a component that detects both changes of a parameter and a distribution value of the parameter. The change detector 40 includes structural components such as a receiver 41, a distribution generator 43, a change determiner 45, and an output 47. In one alternative configuration, the receiver 41 may be external to the change detector 30. Furthermore, in another alternative configuration, additional structural components may be included in the change detector 40 in addition to those illustrated in FIG. 4.The receiver 41 is a structural component that receives values of a parameter from the parameter part 115 of the diagnostic model unit 11 shown in FIG. 1.

The distribution generator 43 generates a probability distribution of the current parameter value.

The change determiner 45 determines the change of the current parameter received from the receiver 41, and determines the change of the probability distribution of the current parameter, which is generated at the distribution generator 43.

The change determiner 45 compares a distribution value, indicative of properties of the probability distribution of the current parameter value, with a distribution value, indicative of properties of a probability distribution of a reference parameter value. After the comparison, the change determiner 45 determines whether the probability distribution of the parameter has changed based on a differential between the distribution value of the current parameter value and the distribution value of the reference parameter value. For example, in one example where a distribution value of the current parameter value has changed from a distribution value of a reference parameter value, such as a default distribution value of a parameter, more than a predetermined extent, in a constant direction for a predetermined period of time, or at a speed or acceleration greater than a predetermined level, the change determiner 45 determines that the probability distribution of the parameter has changed. In response to the determination, the output 47 outputs an alarm signal.

If it is determined that the probability distribution of the parameter has not changed, the change determiner 45 compares the current parameter value received by the receiver 41 with a reference parameter value pre-stored in the change detector 40. After the comparison, the change determiner 45 determines whether the parameter has been changed based on a differential between the current parameter value and the reference parameter value. For example, in a case where the current parameter value has changed from the reference parameter value, such as a default parameter value, more than a predetermined value, in a constant direction for a predetermined period of time, or at a speed or acceleration at or greater than a predetermined level, the change determiner 45 determines that the parameter has been changed. In response to the determination, the output 47 outputs an alarm signal.

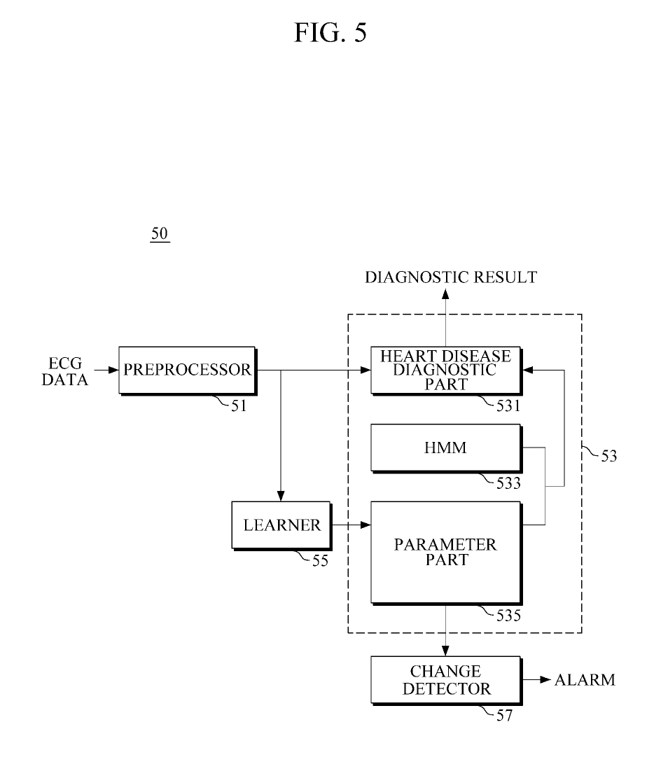

FIG. 5 is a block diagram illustrating another embodiment of a diagnostic apparatus.

Referring to FIG. 5, there is provided an example of a diagnostic system employing a diagnostic model that receives ECG data detected and generated from a subject being observed and performs analysis on the received ECG data using HMM designed to diagnose a heart disease of the subject of observation. The diagnostic system 50 includes components such as a preprocessor 51, a diagnostic model unit 53, a learner 55, and a change detector 57.

The preprocessor 51 converts raw ECG signals to preprocessed signals or values using a transform technique, such as a wavelet transform or a Fourier transform. In addition, the preprocessed signals or values output from the preprocessor 51 are values extracted from the raw ECG signal using, in one example, a signal processing algorithm. For example, the extracted values may be features (e.g., P, Q, R, S, T, and U) or values between features (e.g., P-P or R-R interval and a heart rate).

The learner 55 is a component similar to the learner 13 shown in FIG. 1, which changes parameters of the diagnostic model unit 53 through online learning. The change detector 57 is a component similar to the change detector 15 shown in FIG. 1, which detects changes of parameters in the diagnostic model unit 53. The change detector 57 may be configured to use any one of the change detectors 20, 30, or 40 described above with reference to FIGS. 2, 3 and 4.

The diagnostic model unit 53 receives raw ECG signals or signals processed at the preprocessor 51 and performs diagnosis using HMM as a diagnostic model. The diagnostic model unit 53 includes a heart disease diagnostic part 531, an HMM model structure part 533, and parameter part 535 including a processor with conditional transition probabilities and conditional output probabilities as parameters.

The heart disease diagnostic part 531 receives time-series data of ECG signals detected from a subject of observation and performs diagnosis on the time-series data based on the HMM model structure in the HMM model structure part 533 and the parameters in the parameter part 535 to output a diagnostic result that estimates or predicts the subject's condition regarding a heart disease.

The HMM model structure part 533 includes hidden nodes and observable nodes. Meanwhile, the parameter part 535 includes conditional transition probabilities between a default distribution and time of the hidden nodes, and conditional output probabilities between the hidden nodes and the observable nodes.

In case for HMM designed to diagnose a heart disease based on ECG signals, a value of an observable node is a raw ECG signal. In another example, the value of an observable node is a value preprocessed from the original ECG at the preprocessor 51.

Furthermore, values of hidden nodes includes values indicative of the current state of the heart, a value indicative of medical significance on ECG, or a state that models a health condition. The current state of heart may include atrial systole, complete atrial systole, ventricular systole, complete ventricular systole, ventricular diastolic relaxation, and/or diastasis, and the health condition may include an increasing heart rate, a decreasing heart rate, a high heart rate, a low heart rate, and/or a stable heart rate.

In an example in which a raw ECG signal is used as a value of an observable node and a state that models a body's health condition is used as a value of a hidden node. The body's health condition may include an increasing heart rate, a decreasing heart rate, higher heart rate, a state indicating a lower heart rate, and a stable heart rate, etc.

In such a case, a degree of change of a conditional transition probability of a hidden node in the parameter part 535 may indicate a degree of body respondence to an external environment. If a degree of change of a conditional transition probability indicates a change that shows a stable heart rate increasing or becoming a high heart rate, a rapid increase of a conditional transition probability could mean that a stable heart rate is increased easily or rapidly, thereby leading to a conclusion that the body is responding sensitively to external factors. On the other hand, if a conditional transition probability is rapidly decreasing, such conditional transition probability might indicate that a stable heart rate is decreased easily or rapidly, thereby leading to a conclusion that a body function is beginning to deteriorate. Therefore, a conditional transition probability of a hidden node is changed to a certain extent, slowly increased/decreased, or rapidly increased/decreased, a determination is made that a condition of a patient to be observed has deteriorated or improved or that new diagnosis of the patient needs to be performed by doctors.

In addition, a conditional output probability between a hidden node and an observable node in the parameter part 535 indicates a relationship between a heart rate and a heart condition of a patient at a specific point in time. A degree of change of the conditional output probability may imply a change of relationships between a hidden node and an observable node, which is difficult to identify using a change of values the observable node. For example, when a patient's heart is in a steady state, a heart rate on ECG indicates a baseline heart rate of the patient, and a degree of change in a conditional output probability indicates a change in the baseline heart rate of the patient.

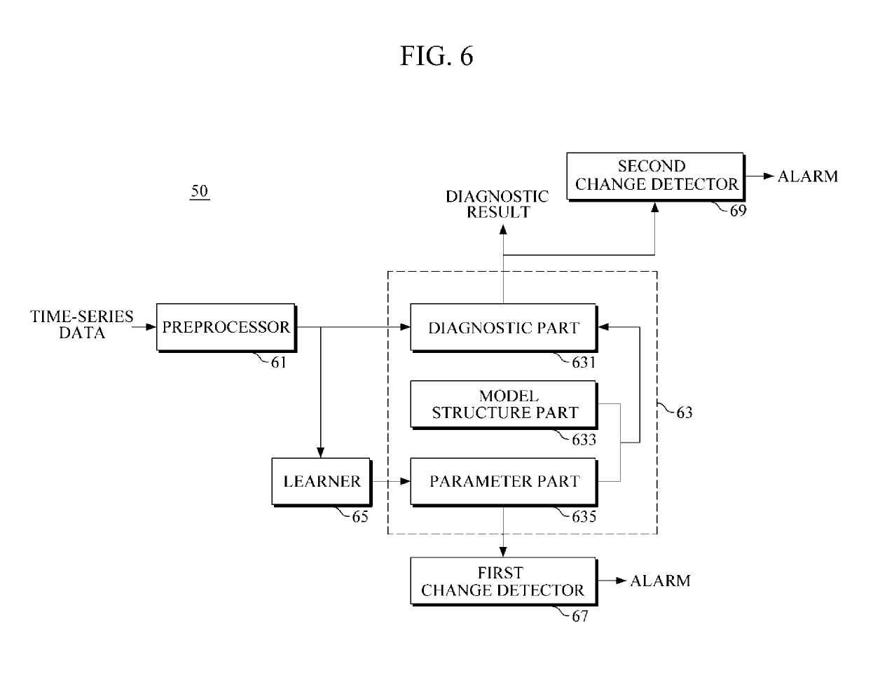

FIG. 6 is a block diagram illustrating another embodiment of a diagnostic apparatus.

Referring to FIG. 6, there is provided an example of a diagnostic apparatus 60 that employs a diagnostic model receiving time-series data generated based on detection from a subject of observation and analyzing the received time-series data. The diagnostic apparatus 60 changes a parameter of the diagnostic model through online learning, detects changes of the parameter, and detects a change of a diagnostic result output from the diagnostic model. The diagnostic system 60 includes a preprocessor 61, a diagnostic model unit 63, a learner 65, a first change detector 67, and a second change detector 69 or the like.

The preprocessor 61, the diagnostic model unit 63, the learner 65, and the change detector 67 are similar to the preprocessor 51, the diagnostic model unit 53, the learner 55, and the change detector 57 according to the embodiment shown in FIG. 5, respectively.

In the example shown in FIG. 6, a diagnostic result output from the diagnostic model unit 63 is an alarm signal that is output from the second change detector 69 in response to a detected parameter change. The second change detector 69 has a structure that is similar to that of the first change detector 67, but the second change detector 69 detects a change of a diagnostic result, whereas the first change detector 67 detects a parameter change.

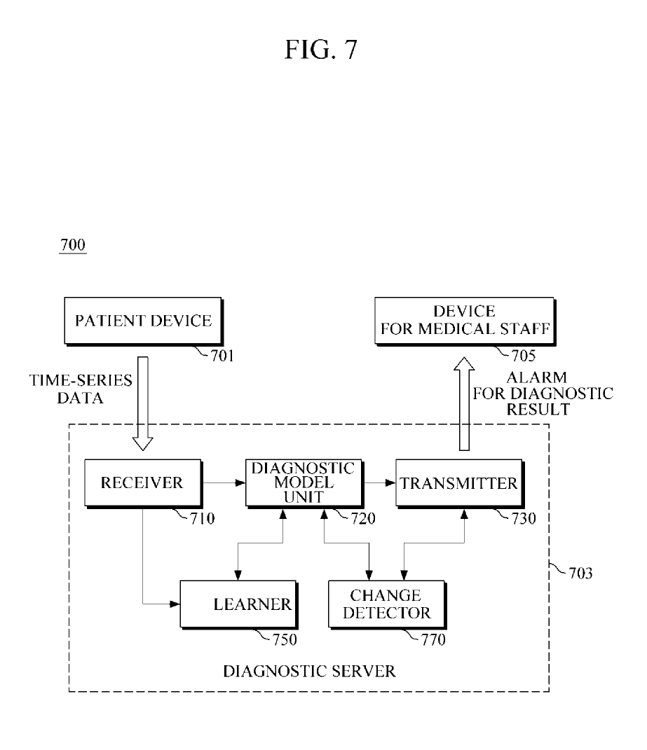

FIG. 7 is a block diagram illustrating another embodiment of a diagnostic apparatus.

Referring to FIG. 7, there is provided an example of a remote diagnostic environment 700 in which a patient device 701, a diagnostic server 703, and a device 705 for medical staff communicate with each other. A diagnostic apparatus, according to an exemplary embodiment, may be included in the diagnostic server 703.

The diagnostic server 703 includes structural components such as a receiver 710, a diagnostic model unit 720, a transmitter 730, a learner 750, and a change detector 770.

The receiver 710 is a structural component that receives time-series data from the patient device 710 over a wired/wireless communication network. Similar to the diagnostic model unit 11 in the diagnostic system shown in FIG. 1, the diagnostic model unit 720 is a model-based diagnosis processing structural component that performs analysis on time-series data based on a probability model and outputs an estimated or predicted result as a diagnostic result. The learner 750 is an online-learning component that changes a parameter in the diagnostic model unit 720 in real time. As described above with reference to FIGS. 2 to 4, the change detector 770 is a structural component that detects a parameter change made through online learning, and outputs an alarm signal. The transmitter 730 is a structural component that transmits a diagnostic result or an alarm signal to the device 705 for medical staff over a wired/wireless communication network.

In the diagnostic environment 700 illustrated in FIG. 7, the patient device 701, the diagnostic server 703, and the device 705 for medical staff may be a structural device, such as a smart phone, a laptop, or a desktop. The patient device 701 acquires ECG signals from an ECG sensor attached on a patient's body. The patient device 701 transmits the ECG signals to the remote diagnostic server 703 over a communication network, such as a wired/wireless Internet. The diagnostic server 703 receives the ECG signals from the patient device 701 and performs diagnosis based on a diagnostic model, such as HMM that is modeled to diagnose a heart disease of a patient. During diagnosis, parameters of a diagnostic model may change through a learning process using the received ECG signals as training data. If a parameter change reaches a certain level, the parameter change is detected, and, in turn, an alarm signal is output. Consecutive diagnostic results from the ECG signals of a patient and an alarm signal in response to detection of the parameter change are transmitted to the remote device 705 for medical staff over a communication network, such as a wired/wireless Internet.

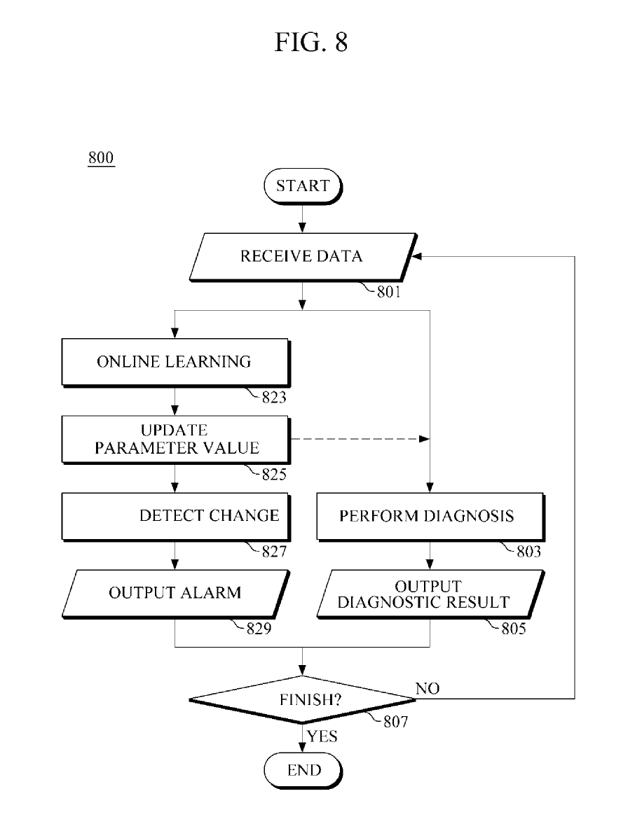

Hereinafter, embodiments of a diagnostic method are described with reference to FIGS. 8 to 13. The diagnostic methods described with reference to FIGS. 8 to 13 are merely illustrative and exemplary. It is apparent for those skilled in the art that different methods with various combinations are possible within the scope of the following claims. All or part of a diagnostic method may be encoded as computer-implementable instructions, modules, software, data, algorithms or procedures performed by a processor of a computing device, a specific task is enabled to be implemented. The computer-implementable instructions may be encoded in a programming language, such as Basic, Fortran, C and C++, and then compiled into machine language.

FIG. 8 is a flow chart illustrating an embodiment of a diagnostic method.

Referring to FIG. 8, a diagnostic method 800 starts out at operation 801 to receive time-series data captured from a subject of observation at a specific point in time. Once the time-series data is received, diagnosis on the received time-series data is performed in operation 803. Operation 803 is a model-based diagnosing operation in which diagnosis is performed based on a model structure and parameters of a diagnostic model designed for probability model-based analysis. Once the diagnosis is first performed, the method 800 learns a default parameter value of the parameter using previously collected training data. After the diagnosis is performed, in operation 805, the method outputs a diagnostic result.

Furthermore, online learning is performed on the received time-series data as training data in operation 823, and accordingly, a parameter value to be used in operation 803 may be updated in operation 825. The updated parameter value is used for diagnosis on data currently received in operation 803 or on data to be received.

In operation 827, by detecting that a change in degree of the parameter value is greater than a predetermined extent by a pre-stored standard, the method 800 detects a parameter change. In operation 829, in response to detection of the parameter change, the method 800 outputs an alarm signal.

In operation 807, the method 800 determines whether data receipt is finished, and, if not, the method 800 waits to receive next data in operation 801. If the next data is received, operations identical to those described above (operations 803 to 807, and 823 to 829) may be performed on the next data.

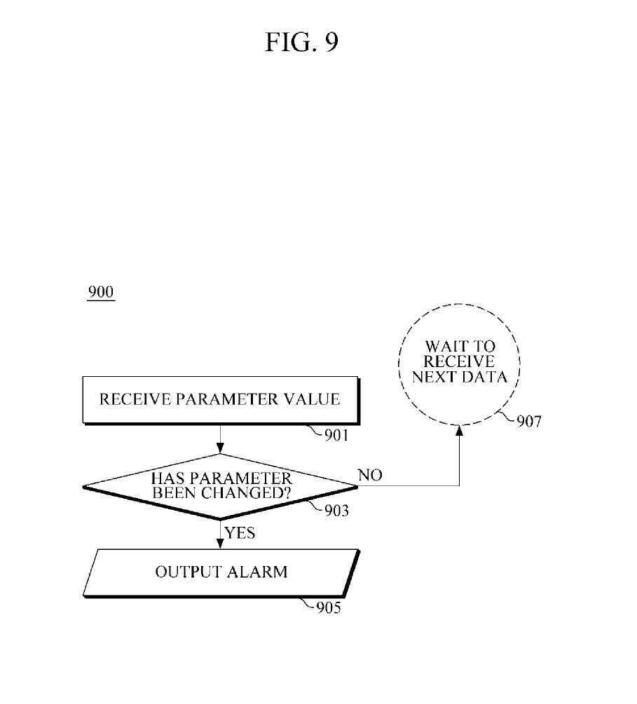

FIG. 9 is a flow chart illustrating an embodiment of a method shown in FIG. 8, in which a parameter change is detected.

Referring to FIG. 9, a method 900 for detecting a parameter change is an operation for detecting a change in a parameter value and starts out by operation 901 for receiving a parameter value changed by online learning.

In operation 903, the method 900 compares currently received parameter value with a pre-stored reference parameter value, and determines whether the parameter has been changed based on a differential between the currently received parameter value and the reference parameter value.

Herein, the reference parameter value may be identical to a default parameter value of a diagnostic model, which is set before the learner performs online learning on the parameter. In the case in which a parameter of a diagnostic model is designed for diagnosis of a disease, a default parameter value of the diagnostic model is learned using training data about a healthy condition.

In operation 903, if a differential between the currently-received parameter value and the reference parameter value is greater than a predetermined extent, in operation 905, the method determines that a parameter change is detected (which corresponds to “YES” in operation 903), and then, an alarm is output. Alternatively, if a differential between the currently-received parameter value and the reference parameter value is smaller than a predetermined extent, in operation 907, the method 900 determines that the parameter has been changed within a tolerance range, and thus the parameter change is not detected (which corresponds to “NO” in operation 903), and waits for next data to be received.

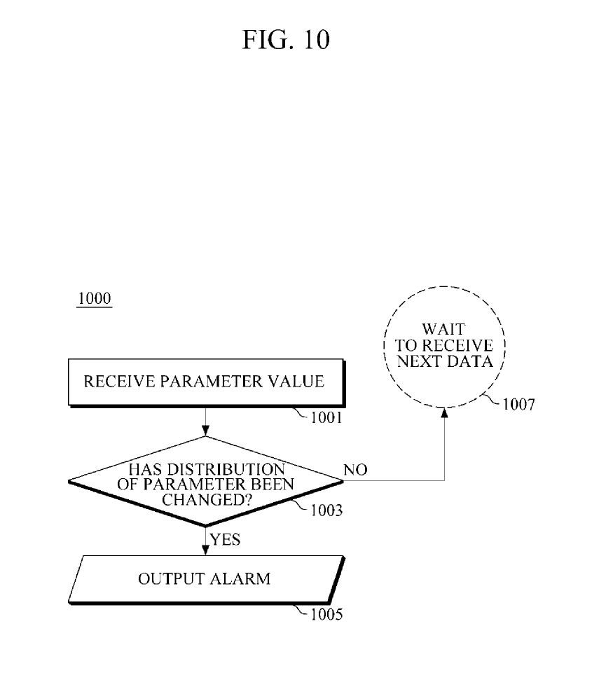

FIG. 10 is a flow chart illustrating another embodiment of a method shown in FIG. 8, in which a parameter change is detected.

Referring to FIG. 10, a method 1000 to detect a parameter change is implemented by detecting a change in a probability distribution of a parameter. In operation 1001, the method 1000 receives a parameter value changed through online learning.

Then, in operation 1003, the method 1000 calculates a probability distribution of the currently received parameter value and compares the probability distribution with a probability distribution of a pre-stored reference parameter value. That is, the method 1000 calculates a distribution value of the currently received parameter, such as a mean indicating properties of the probability distribution of the currently received parameter value or a variance of the currently received parameter, compares the distribution value with the reference parameter distribution value, such as a mean of the probability distribution of the reference parameter value or a variance of the reference parameter value. Then, in operation 1003, the method 1000 determines whether a parameter distribution has been changed based on a differential between the currently received parameter distribution value and the reference parameter distribution value.

In one example, the reference parameter distribution value may be identical to a default parameter distribution value of a diagnostic model, which was set before the learner performed online learning on the parameter.

In operation 1003, if a differential between the currently received parameter distribution value and the reference parameter distribution value is greater than a predetermined extent, at operation 1005, the method 1000 determines that a change in a distribution of a corresponding parameter is detected (which corresponds to “YES” in operation 1003), and thus, an alarm is output. Alternatively, if a differential between the distribution value of the currently received parameter and the distribution value of the reference parameter value is smaller than a predetermined extent, at operation 1007, the method 1000 determines that the parameter has been changed within a tolerance range, and thus, a change in a distribution of a corresponding parameter is not detected (which corresponds to “NO” in operation 1003, thereby waiting for next data to be received.

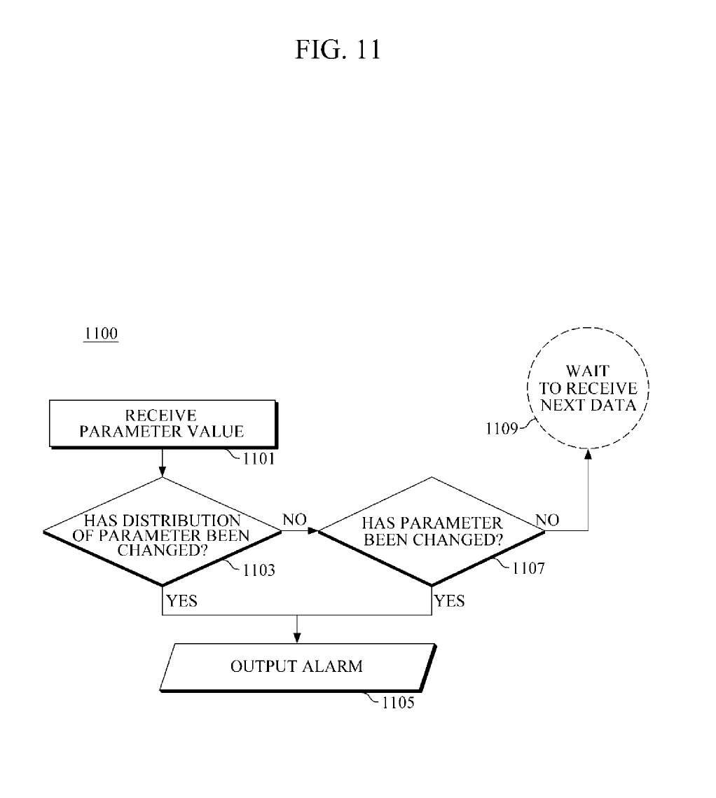

FIG. 11 is a flow chart illustrating another embodiment of a method shown in FIG. 8, in which a parameter change is detected.

Referring to FIG. 11, method 1100 is implemented by detecting both a parameter change and a change in a probability distribution of a parameter. In operation 1101, the method 1100 receives a parameter value changed by online learning.

Then, in operation 1103, the method 1100 detects a change in a distribution of a parameter and, in operation 1107, the method 1100 detects whether a parameter has changed.

In operation 1103, the method 1100 calculates a probability distribution of the currently received parameter value, which is then compared with a probability distribution of a pre-stored reference parameter value. For example, whether a distribution of a parameter has been changed is determined based on a differential between a distribution value of the currently received parameter and a distribution value of the reference parameter.

In operation 1103, if a differential between a distribution value of the currently received parameter and a distribution value of the reference parameter is greater than a predetermined extent, the method 1100 determines that a change in a parameter distribution is detected (which corresponds to “YES” in operation 1103), and thus, in operation 1105, the method 1100 outputs an alarm. Alternatively, if a differential between a distribution value of the currently received parameter and a distribution value of the reference parameter is less than a predetermined extent, the method 1100 determines that a parameter has been changed within a tolerance range and thus a change of a distribution of the parameter is not detected (which corresponds to “NO” in operation 1103), and proceeding to operation 1107 to detect a parameter change.

In operation 1107, the method 1100 compares the currently received parameter value with a pre-stored reference parameter value, and determines a parameter change based on a differential between the currently received parameter value and the reference parameter value.

In operation 1107, if a differential between the currently received parameter value with the reference parameter value is greater than a predetermined extent, the method 1100 determines that a parameter change is detected (which corresponds to “YES” in operation 1107), and, outputs an alarm in operation 1105. Alternatively, if a differential between the currently received parameter value with the reference parameter value is lesser than a predetermined extent, the method 1100 determines that a parameter has been changed within a tolerance range, and thus, a change in the parameter is not detected (which corresponds to “NO” in operation 1107), and waits for next data to be received in operation 1109.

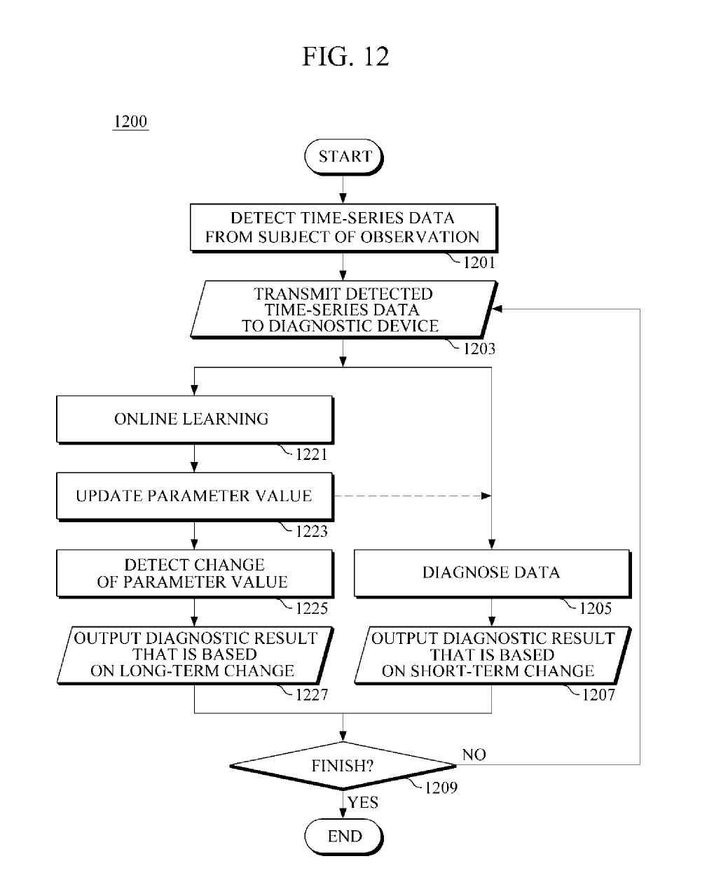

FIG. 12 is a flow chart illustrating another embodiment of a diagnostic method.

Referring to FIG. 12, a diagnostic method 1200 starts out with operation 1201 in which time-series data captured from a subject of observation at a specific point in time are detected. At operation 1203, the method 1200 transmits the detected time-series data to a remote diagnostic device over a communication network. In response to receipt of the time-series data, in operation 1205, the method 1200 performs, through the diagnostic device, diagnosis on the received time-series data. Operation 1205 is a model-based diagnosing operation in which diagnosis is performed based on a model structure and parameters of a diagnostic model designed for probability model-based analysis. When the diagnosis is first performed, a default parameter value of the parameter may be learned using previously collected training data. After the diagnosis is performed, in operation 1207, the method 1200 outputs a diagnostic result. The diagnostic result output in operation 1207 is a diagnostic result according to a relatively short-term data change.

Furthermore, in operation 1221, the method 1200 performs online learning using the received time-series data as training data, and accordingly, in operation 1223, the method 1200 updates a parameter value to be used in operation 1205. The method 1200 uses the updated parameter value for diagnosis of the time-series data currently received in operation 1205 or for diagnosis of data to be received.

Then, by detecting that a change in degree of the parameter value is greater than a predetermined extent by a pre-stored standard, in operation 1225, the method detects a parameter change. If the parameter change is detected, in operation 1227, the method 1200 outputs an alarm signal, wherein the alarm signal is a diagnostic result based on a relatively long-term data change.

In operation 1209, the method 1200 determines whether data receipt is complete, and, if not, in operation 1203, the method 1200 waits for next data to be received. If the next data is received, operations identical to those described above (operations 1205 to 1207, and 1221 to 1227) may be performed on the next data.

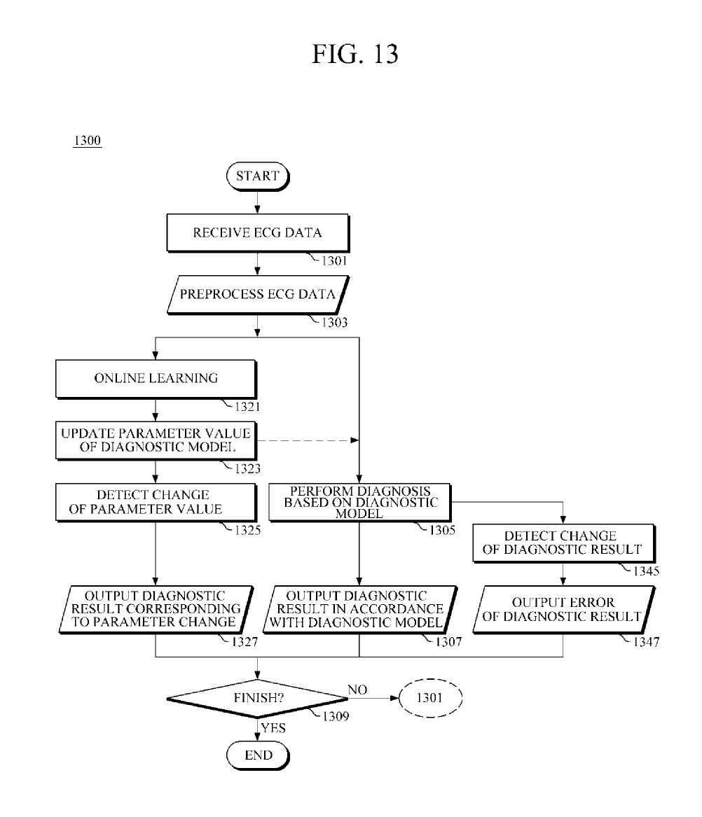

FIG. 13 is a flow chart illustrating another embodiment of a diagnostic method.

Referring to FIG. 13, in a diagnostic method 1003, in operation 1301, ECG data is received as time-series data detected from a subject of observation detected at a specific point in time. The received ECG data is preprocessed using a transform technique, such as wavelet transform or Fourier transform. In operation 1305, the method 1300 performs diagnosis on the preprocessed data based on a diagnostic mode, such as HMM that is modeled to diagnose, for example, a cardiovascular disease. Operation 1305 is a model-based diagnosing operation in which diagnosis is performed based on a model structure and parameters of a diagnostic model designed for probability model-based analysis. When the diagnosis is first performed, a default parameter value of the parameter is learned using previously collected training data. After the diagnosis is performed, in operation 1307, the method 1300 outputs a diagnostic result. The diagnostic result output in operation 1307 may include an estimated or predicted value indicative of a state modeled by the diagnostic model.

Online learning is performed using pre-processed ECG data as training data in operation 1323, and accordingly, in operation 1323, the method 1300 updates a parameter value to be used in operation 1305. The updated parameter is used in operation 1305 in which the currently received data or next data to be received are diagnosed.

In operation 1325, by detecting that a change in degree of the parameter value is greater than a predetermined extent by a pre-stored standard, the method 1300 detects a parameter change. If the parameter change is detected, in operation 1327, the method 1300 outputs a diagnostic result, wherein an alarm signal is output as the diagnostic result.

In operation 1345, the method 1300 detects a change of a diagnostic result output from the diagnostic model. The detection of the change of the diagnostic result may be possible using, for example, CUSUM algorithm. After the detection of the change of the diagnostic result, in operation 1347, the method 1300 outputs an alarm to notify that the diagnostic result is an error.

In operation 1309, the method 1300 determines whether data receipt is complete, and, if not, in operation 1301, the method 1300 waits to receive next data. In response to receipt of the next data, operations identical to those described above (operations 1305 to 1307, 1345 to 1347, and 1321 to 1327) may be performed on the next data.

It is to be understood that in the embodiment of the present invention, the operations in FIGS. 8-13 are performed in the sequence and manner as shown although the order of some operations and the like may be changed without departing from the spirit and scope of the described configurations. In accordance with an illustrative example, a computer program embodied on a non-transitory computer-readable medium may also be provided, encoding instructions to perform at least the method described in FIGS. 8-13.

Program instructions to perform a method described in FIGS. 8-13, or one or more operations thereof, may be recorded, stored, or fixed in one or more computer-readable storage media. The program instructions may be implemented by a computer. For example, the computer may cause a processor to execute the program instructions. The media may include, alone or in combination with the program instructions, data files, data structures, and the like. Examples of computer-readable media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable recording mediums. Also, functional programs, codes, and code segments for accomplishing the example embodiments disclosed herein may be easily construed by programmers skilled in the art to which the embodiments pertain based on and using the flow diagrams and block diagrams of the figures and their corresponding descriptions as provided herein.

The parts, units, and apparatuses described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processor.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A diagnostic apparatus, comprising:
a diagnostic model unit configured to diagnose time-series data based on a model structure and parameters of a diagnostic model performing probability model-based analysis;
a learner configured to change the parameters using the time-series data as training data; and
a change detector configured to detect a parameter change and output an alarm signal based on the detected parameter change.

2. The diagnostic apparatus of claim 1, wherein the change detector further comprises
a receiver configured to receive a parameter value of a parameter,
a change determiner configured to compare the parameter value with a pre-stored reference parameter value and determine whether the parameter has changed based on a difference between the parameter value and the pre-stored reference parameter value, and
an output configured to output the alarm signal in response to a determination that the parameter has changed.

3. The diagnostic apparatus of claim 2, wherein the pre-stored reference parameter value is identical to a default parameter value, which is pre-set prior to the learner performing online learning on the parameter.

4. The diagnostic apparatus of claim 2, wherein, in response to the parameter value changing from a default parameter set before the learner performs online learning, the change determiner is further configured to determine that the parameter changes to be greater than a predetermined value, in a constant direction for a predetermined period of time, or at a speed or acceleration greater than a predetermined level.

5. The diagnostic apparatus of claim 2, wherein the parameter value gradually or suddenly changes from a default value or an initial value through online learning.

6. The diagnostic apparatus of claim 1, wherein the change detector further comprises
a receiver configured to receive the parameter value,
a distribution generator configured to generate a probability distribution of the parameter value,
a change determiner configured to determine whether a probability distribution of the parameter value has changed based on a difference between a distribution value indicative of properties of the probability distribution of the parameter value and a distribution value indicative of properties of a probability distribution of the reference parameter value, and
an output configured to output the alarm signal in response to a determination that the probability distribution of the parameter value has changed.

7. The diagnostic apparatus of claim 6, wherein the probability distribution of the reference parameter value is identical to a probability distribution of a default parameter value pre-set before the learner performs online learning.

8. The diagnostic apparatus of claim 6, wherein, in response to a mean or a variance of the probability distribution of the parameter value changing from a mean or a variance of the probability distribution of the default parameter value more than a predetermined value, the change determiner is further configured to determine that the probability distribution of the parameter value changes in a constant direction for a predetermined period of time, or at a speed greater than a predetermined level.

9. The diagnostic apparatus of claim 1, wherein the diagnostic model unit is further configured to perform diagnosis based on
a model structure configured to comprise hidden nodes and observable nodes, and
parameters, each comprising a conditional transition probability for a default distribution of the hidden nodes relative to time and a conditional output probability for a relation between the hidden nodes and the observable nodes.

10. The diagnostic apparatus of claim 9, wherein
the diagnostic model unit is configured to receive electrocardiography (ECG) signals detected from a subject of observation as time-series data and output a diagnostic result estimating or predicting a heart disease of the subject based on the received ECG signals,
values of the observable nodes comprises a raw ECG signal, a value converted from the raw ECG signal, or a value extracted from the ECG raw signal, and
values of the hidden nodes comprises a value indicative of a heart condition, a value indicative of medical significance presented on ECG, or a state of a body condition, wherein the value indicative of the heart condition comprises atrial systole, complete atrial systole, ventricular systole, complete ventricular systole, ventricular diastolic relaxation, and diastasis, or the body condition comprises an increasing heart rate, a decreasing heart rate, a high heart rate, a low heart rate, or a stable heart rate.

11. The diagnostic apparatus of claim 1, wherein the time-series data is transmitted from a remote device to the diagnostic model unit over a communication network, or a diagnostic result output from the diagnostic model unit is transmitted to the remote device over the communication network.

12. The diagnostic apparatus of claim 1, wherein the diagnostic model unit comprises
a diagnostic part,
a model structure part, and
a parameter part, wherein the diagnostic part is configured to perform diagnosis based on a model structure pre-stored in the model structure part and a parameter pre-stored in the parameter part, wherein the parameter is used to obtain a diagnostic result in accordance with the model structure in the model structure part.

13. The diagnostic apparatus of claim 12, wherein the model structure comprises a structure indicative of a correlation between the observable nodes and the hidden nodes.

14. The diagnostic apparatus of claim 12, wherein initial values of the parameter part are values learned using a predefined training data.

15. The diagnostic apparatus of claim 12, wherein the diagnostic model unit performs diagnosis on data received at a present time or a next time using a parameter changed at the present time through online learning.

16. A diagnostic method comprising:
diagnosing received time-series data based on a model structure and parameters of a diagnostic model performing probability model-based analysis;
performing online learning by changing the parameters in real time using the received time-series data as training data; and
detecting a parameter change and outputting an alarm signal based on the detected parameter change.

17. The diagnostic method of claim 16, wherein the detecting of a parameter change comprises
receiving a parameter value of a parameter,
comparing the parameter value with a pre-stored reference parameter value, and determining whether the parameter has changed based on a difference between the parameter value and the pre-stored reference parameter value, and
in response to a determination that the parameter has changed, outputting the alarm signal.

18. The diagnostic method of claim 17, wherein the pre-stored reference parameter value is identical to a default parameter value that is pre-set prior to the online learning is performed on the parameter.

19. The diagnostic method of claim 17, wherein the determining of a parameter value comprises
in response to the parameter value changing from a default parameter value set before online learning is performed, determining that the parameter changes to be greater than a predetermined value, in a constant direction for a predetermined period of time, or at a speed or acceleration greater than a predetermined level.

20. The diagnostic method of claim 16, wherein the detecting of a parameter change comprises
receiving a parameter value of a parameter,
generating a probability distribution of the parameter value,
determining whether the probability distribution of the parameter value has changed, based on a difference between a distribution value indicative of properties of the probability distribution of the parameter value and a distribution value indicative of properties of a probability distribution of a pre-stored reference parameter value, and
in response to a determination that the probability distribution of the parameter value changing, outputting the alarm signal.

21. The diagnostic method of claim 20, wherein the probability distribution of the pre-stored reference parameter value is identical to a probability distribution of a default parameter value pre-set before online learning performs on the parameter.

22. The diagnostic method of claim 20, wherein the determining whether the probability distribution of the parameter value has changed comprises
in response to a mean or a variance of the probability distribution of the parameter value changing from a mean or a variance of the probability distribution of the default parameter value set, prior to online learning being performed, more than a predetermined value, determining the probability distribution of the parameter value changes in a constant direction for a predetermined period of time, or at a speed or acceleration greater than a predetermined level.

23. The diagnostic method of claim 16, wherein the performing of diagnosis based on the diagnostic model comprises
performing diagnosis based on a model structure configured to comprise hidden nodes and observable nodes, and parameters, each comprising a conditional transition probability of a default distribution of the hidden nodes over time and a conditional output probability between the hidden nodes and the observable nodes.

24. The diagnostic method of claim 23, wherein
the diagnostic model unit is configured to output a diagnostic result estimating or predicting a state of a heart disease of a subject of observation based on Electrocardiography (ECG) signals detected from the subject,
values of the observable nodes comprises a raw ECG signal, a value converted from the raw ECG signal, or a value extracted from the raw ECG signal, and
values of the hidden nodes comprises a value indicative of a heart condition, a value indicative of medical significance on ECG, or a state that models a body condition, wherein the heart condition comprises atrial systole, complete atrial systole, ventricular systole, complete ventricular systole, ventricular diastolic relaxation, and diastasis, or the body condition comprises an increasing heart rate, a decreasing heart rate, a high heart rate, a low heart rate, or a stable heart rate.

25. The diagnostic method of claim 16, wherein the time-series data is transmitted from a device at a remote location to the diagnostic model unit over a communication network, or a diagnostic result output from the diagnostic model unit is transmitted to the device at a remote location to another device over a communication network.
